(19) United States
(12) Patent Application Publication
(10) Pub. No.: US 2020/0268322 A1
(43) Pub. Date: Aug. 27, 2020

(54) ANALYTE SENSOR COVER

(71) Applicant: Chris A. Moxon, Lakewood, CA (US)

(72) Inventor: Chris A. Moxon, Lakewood, CA (US)

(21) Appl. No.: 16/287,351

(22) Filed: Feb. 27, 2019

(51) Int. Cl. A61B 5/00; A61B 5/145

(52) U.S. Cl. CPC A61B 5/6833; A61B 2560/0412; A61B 5/14546

(57) ABSTRACT

An analyte sensor cover is disclosed comprising a sensor cover including a top side, a bottom side, and an aperture. The bottom side includes a recessed portion dimensioned to at least partially receive an in vivo analyte sensor. An adhesive component adheres to the top side of the sensor cover and at least partially to the skin of a user to retain the in vivo analyte sensor in a suitable position.

### CROSS-REFERENCE TO RELATED APPLICATIONS

This application claims priority to U.S. Provisional Application 62/725,140 entitled “SENSOR COVER” and filed Aug. 30, 2018, which is hereby incorporated by reference in its entirety.

### TECHNICAL FIELD

The embodiments generally relate to biometric sensor components and more specifically relate to analyte sensor covers for analyte sensors in communication with an analyte monitor.

### BACKGROUND

Diabetes is a group of metabolic disorders in which there are high blood sugar levels over a prolonged period causing a variety of complications if left untreated. The CDC reports that now more than 100 million Americans are living with diabetes or are considered prediabetic and at risk of developing the disorder. Diabetes is a chronic disease for which there is no known cure. Management of the disease consists of maintaining blood sugar levels at or near a normal level. To monitor blood sugar levels, many patients utilize a continuous glucose monitor that can alert the patient of fluctuations in blood glucose levels. The patient may then use this information to determine if and when insulin is needed to reduce glucose levels in their body or when additional glucose is needed to raise the level of glucose in their body.

Abbott Diabetes Care, Inc. has created a blood glucose monitoring system named FreeStyle Libre (US20160183854A1). It is made up of a sensor and an electronic device to wirelessly read and display the data produced by the sensor. The sensor sticks to the skin with an adhesive and provides continuous blood glucose data for a period of about 10 days.

A common problem is that the sensor can be partially dislodged by everyday activities and once it is partially dislodged the sensor will no longer function correctly and needs to be replaced before the expiration of its useful life. This can be very costly for patients if this happens regularly. The dislodging of the device can occur while showering, sleeping, changing clothing, inadvertent contact, exercising, and many other daily tasks.

One method to better secure the sensor is to apply adhesive tape to the sensor and the adjacent skin. The problem with this method is that the tape will need to be replaced once or twice during the life of the sensor, and it is almost impossible to remove the tape from the sensor without dislodging it.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The embodiments described herein relate to an analyte sensor cover comprising a sensor cover including a top side, a bottom side, and an aperture. The bottom side includes a recessed portion dimensioned to at least partially receive an in vivo analyte sensor. An adhesive component adheres to the top side of the sensor cover and at least partially to the skin of a user to retain the in vivo analyte sensor in a suitable position.

In one aspect, the recessed portion has a height sufficient to prevent the sensor cover's contact with the skin.

In one aspect, the adhesive component is a kinesiology tape. The kinesiology tape may be provided in precut sections or as a roll which is cut by the user. The adhesive component may be provided having rounded edges to reduce the likelihood of accidental removal.

In one aspect, the adhesive component is arranged to contact the sensor cover and not the in vivo analyte sensor.

In one aspect, the adhesive component is further comprised of a second aperture positioned to align with the first aperture of the sensor cover.

In one aspect, the adhesive component is substantially waterproof and sweatproof.

In one aspect, the sensor cover is reusable for a plurality of in vivo analyte sensors.

In one aspect, the interface between the in vivo analyte sensor and the analyte monitor permits collection of raw sensor data using a sharp.

In one aspect, the distance between the recessed portion and the annular rim is less than ⅛ inch.

In one aspect, a method for retaining an analyte sensor in a suitable position on a user includes the steps of embedding the in vivo analyte sensor in the user's skin and positioning the sensor cover over the in vivo analyte sensor. The sensor cover is positioned such that the bottom surface having the recessed portion contacts the upper surface of the analyte sensor. The user then places the adhesive component over the top surface of the sensor cover such that the in vivo analyte sensor is retained in the suitable position to continuously monitor analytes in the blood of the user.

### BRIEF DESCRIPTION OF THE DRAWINGS

A complete understanding of the present invention and the advantages and features thereof will be more readily understood by reference to the following detailed description when considered in conjunction with the accompanying drawings wherein:

FIG. 1 illustrates a perspective view of the analyte sensor and sensor cover, according to some embodiments;

FIG. 2 illustrates a perspective view of the bottom side of the sensor cover, according to some embodiments;

FIG. 3 illustrates a perspective view of the top side of the sensor cover, according to some embodiments;

FIG. 4 illustrates an analyte sensor and sensor cover positioned on a user, according to some embodiments;

FIG. 5 illustrates an exploded view of the analyte sensor, sensor cover, and an adhesive component, according to some embodiments; and

FIG. 6 illustrates a flowchart of a method for retaining an analyte sensor in a suitable position on a user, according to some embodiments.

### DETAILED DESCRIPTION

The specific details of the single embodiment or variety of embodiments described herein are to the described apparatus and methods of use. Any specific details of the embodiments are used for demonstration purposes only and not unnecessary limitations or inferences are to be understood therefrom.

Before describing in detail exemplary embodiments, it is noted that the embodiments reside primarily in combinations of components related to the analyte sensor cover.

As used herein, relational terms, such as “first” and “second”, “top” and “bottom” and the like, may be used solely to distinguish one entity or element from another entity or element without necessarily requiring or implying any physical or logical relationship or order between such entities or elements.

In general, the embodiments described herein relate to an analyte sensor cover configured to retain an in vivo analyte sensor in the skin of the user. The analyte sensor is partially embedded in the skin of the user to continuously monitor blood analyte levels, such as blood glucose. Once embedded in the user's skin, the analyte sensor is susceptible to dislodging, which causes pain, discomfort, and may require the patient to expend resources to engage a new sensor. The present embodiments provide a means for retaining the in vivo analyte sensor in the patient over a period of time for which the in vivo analyte sensor functions. Commonly, this period is 10-14 days. The sensor cover is dimensioned to retain the in vivo analyte sensor in a suitable position, and it is held in place by an adhesive component.

As used herein, the term “In vivo analyte sensor” includes continuous analyte monitoring systems and flash analyte monitoring systems that can broadcast data from an analyte monitor to various electronic devices automatically. In vivo analyte sensors may operate without the need for finger stick calibration. The term “in vivo” is used to denote the collection of the test sample (i.e., the blood) from inside the body, rather than ex vivo. One skilled in the arts will appreciate that the capabilities of the embodiments may be extended to monitor in vitro samples.

In some embodiments, the in vivo analyte sensor may operate in concert with a drug delivery device (e.g., insulin pumps, or the like). A communication link (e.g., a cable or wireless links such as a radio frequency or the like) may be provided for the transfer of data from the in vivo analyte sensor the drug delivery device. In such embodiments, samples may be collected by scanning the in vivo analyte sensor, or by other means known in the arts.

The embodiments provide a sensor cover acting as a protective shield around the in vivo analyte sensor, helping to ensure it is not dislodged when bumped via everyday activities. The cover is held in place by an adhesive component which is also applied to the surrounding skin. When replacing the adhesive component, the sensor cover easily comes off still attached to the tape making it easy to remove the tape from the sensor cover once it is no longer attached to the skin.

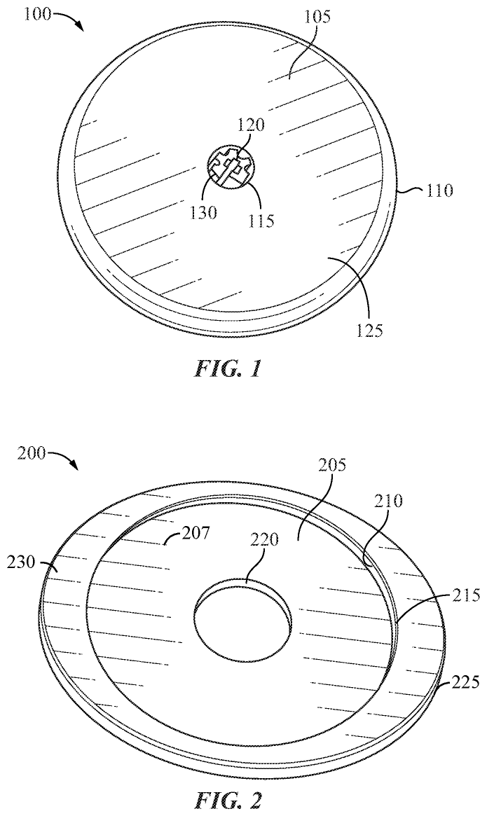

FIG. 1 illustrates the in vivo analyte sensor 100 from a top perspective view. The in vivo analyte sensor 100 is comprised of a top surface 105 terminating in a perimeter 110. The top surface 105 has an aperture 115 which at least partially exposes a reader 120 which broadcasts the sensor data to an analyte monitor (not shown), computing device, or similar receiver configured to display analyte data to the patient. In some embodiments, receptacle 130 may permit the interface between the in vivo analyte sensor 100 and the analyte monitor. The opposite side of this in vivo analyte sensor contains adhesive and a small needle which protrudes into the skin. The in vivo analyte sensor is attached to the skin by the adhesive pad included with the in vivo analyte sensor.

FIG. 2 illustrates the sensor cover 200 having a bottom side 205 comprised of a recessed portion 210 surrounded by an annular rim 215 terminating in an outer edge 225. At least one aperture 220 is positioned at the central axis of the sensor cover 200 to permit an interface to the in vivo analyte sensor when the sensor cover 200 is engaged. The recessed portion 205 defines a first surface having a thickness less than the thickness of the annular rim 215. The recessed portion is dimensioned to at least partially receive the perimeter of the in vivo analyte sensor to facilitate the retainment of the in vivo analyte sensor in a suitable position. The term “suitable position” may be defined as the positions in which the in vivo analyte sensor can collect samples of the blood to transmit data related to blood analyte levels (e.g., blood glucose).

In some embodiments, the distance between the surface 207 of the recessed portion 205 and upper surface 230 of the annular rim 215 is less than ⅛ inch. In a preferred embodiment, the distance between the surface 207 of the recessed portion 205 and upper surface 230 of the annular rim 215 is about 1/16 inch. The surface of the recessed portion 205 contacts with the top surface of the in vivo analyte sensor. The annular rims upper surface 230 is outside the diameter of the in vivo analyte sensor partially embedded in the recessed portion 205 but does not contact the skin of the user as the height of the in vivo analyte sensor is ⅛ inch or greater.

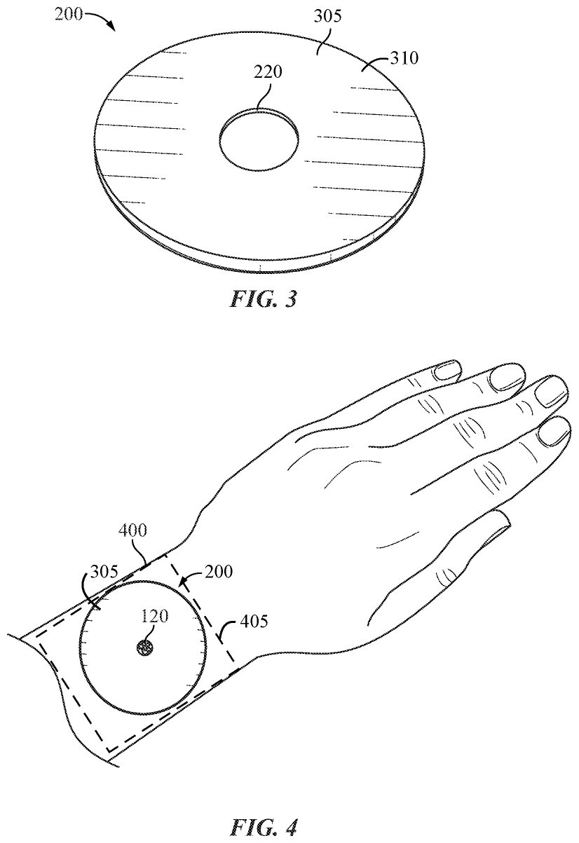

FIG. 3 shows the top side 305 of the sensor cover 200. Adhesive component is applied to the top surface 310 of the sensor cover 200 and to the adjacent surfaces of the skin to hold it in place. FIG. 4 illustrates the sensor cover 200 positioned over the in vivo analyte sensor 100 on a user 400 in an in-use configuration. The user 400 then positions an adhesive component to contact the top surface 310 of the sensor cover 200 and the surrounding skin 405 to retain the in vivo analyte sensor in a suitable position. The reader 120 may still be accessed and is capable of transmitting blood analyte data when in the in-use configuration.

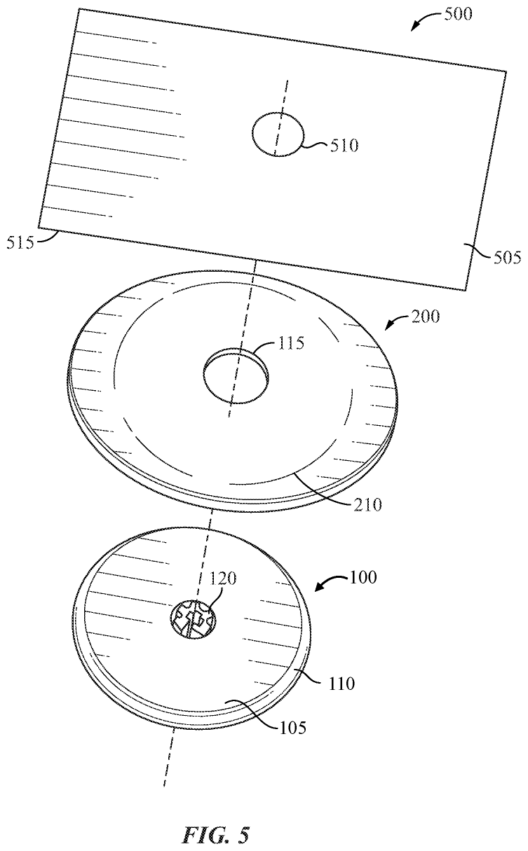

FIG. 5 illustrates an exploded view of the sensor cover system 500 including the adhesive component 505. In some embodiments, the adhesive component 505 may have a central aperture 510 to align with the aperture 115 of the sensor cover 200 such that the reader 120 of the in vivo analyte sensor 100 is exposed. An outer edge 515 of the adhesive component 505 may be provided with rounded edges. The top surface 105 contacts the surface of the recessed portion 210 of the sensor cover 200. The perimeter 110 is received within the recessed portion 210 such that the sensor cover 200 does not contact the skin of the user to avoid complications such as irritation or an allergic response.

In some embodiments, the outer edge 515 may be defined by the user cutting a larger section of adhesive component 505 into a desired size or shape. Rounded edges may aid in preventing the accidental tearing of the adhesive component 505 away from the skin during use. In another embodiment, the adhesive component 505 is provided as a pre-cut adhesive component.

In some embodiments, the sensor cover can be made of various types of material. The aperture 220 in the center of the sensor cover may not exist, and instead of the top surface 310 being solid it may be perforated containing dozens of apertures of around 1/16″ where the holes are separated by ⅛″.

In some embodiments, the sensor cover may be a reusable sensor cover used with a plurality of in vivo analyte sensors in series (i.e., for multiple 10-14-day periods). Another embodiment may be a disposable cover made of a thinner plastic where the surface contains a larger surface area and is covered with adhesive. In this case, the distance between the recessed portion and the annular rim would need to be slightly larger than the ⅛″ thickness of the sensor. Whereas the preferred embodiment uses adhesive tape to hold it in place, the disposable version would not require adhesive tape if the adhesive is attached to the upper surface 230 of the annular rim 215.

The in vivo analyte sensor 100 is adapted to be at least partially inserted into the body of the user, where it can make fluid contact with that user's body fluid (e.g., interstitial fluid (ISF), dermal fluid, or blood) and be used, along with in vivo analyte monitor circuitry, to measure analyte-related data of the user.

The adhesive component 505 can include any adhesive known in the arts. Preferentially, the adhesive component is a kinesiology tape or similar tape that is sufficiently waterproof, sweatproof, and malleable to maintain a sufficient tack during daily activities.

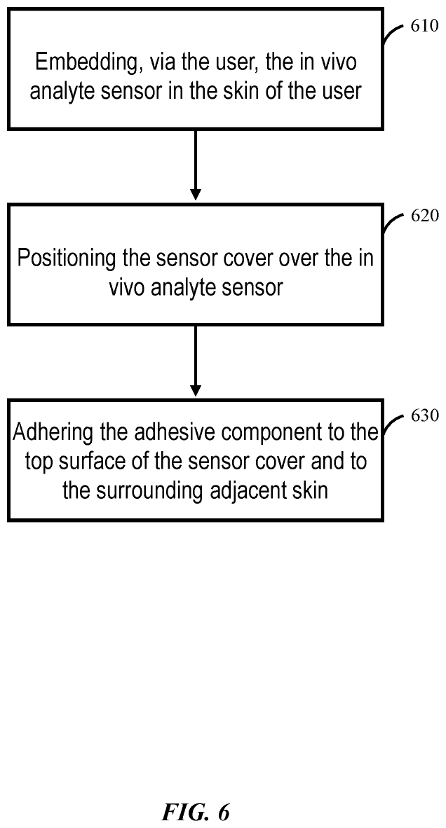

FIG. 6 illustrates a flowchart of a method for retaining an in vivo analyte sensor in a suitable position on a user. In step 610, the user at least partially embeds the in vivo analyte sensor in their skin in suitable position to collect sample data. In step 620, the user positions the sensor cover over the in vivo analyte sensor. In step 630, the user adheres the adhesive component to the upper surface of the sensor cover and to the surrounding adjacent skin to retain the in vivo analyte sensor in the suitable position.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. An analyte sensor cover, comprising:
a sensor cover including a top side and a bottom side and an aperture, the bottom side comprised of a recessed portion dimensioned to at least partially receive an in vivo analyte sensor; and
an adhesive component adhered to the top side of the sensor cover and at least partially to the skin of a user and arranged to retain the in vivo analyte sensor in a suitable position.

2. The analyte sensor cover of claim 1, wherein the sensor cover is further comprised of a first aperture dimensioned to permit an interface between an analyte monitor and the in vivo analyte sensor.

3. The analyte sensor of claim 2, wherein the recessed portion has a height sufficient to prevent the sensor cover's contact with the skin.

4. The analyte sensor cover of claim 3, wherein the adhesive component is a kinesiology tape.

5. The analyte sensor cover of claim 4, wherein the adhesive component is comprised of rounded edges.

6. The analyte sensor of claim 5, wherein the adhesive component is arranged to contact the sensor cover and not the in vivo analyte sensor.

7. The analyte sensor of claim 6, wherein the adhesive component is further comprised of a second aperture positioned to align with the first aperture of the sensor cover.

8. The analyte sensor 1, wherein the sensor cover is reusable for a plurality of in vivo analyte sensors.

9. An analyte sensor cover, comprising:
a sensor cover including a top side and a bottom side and a first aperture, the bottom side comprised of a recessed portion and an annular rim around the recessed portion having a height to partially receive an in vivo analyte sensor;
a first aperture positioned at a central axis of the sensor cover to partially expose the in vivo analyte sensor to permit an interface between the in vivo analyte sensor and an analyte monitor;
an adhesive component adhered to the top side of the sensor cover and at least partially to the skin of a user and arranged to retain the in vivo analyte sensor in a suitable position, the adhesive component including a second aperture aligned with the first aperture.

10. The analyte sensor cover of claim 9, wherein the interface permits collection of raw sensor data using a sharp.

11. The analyte sensor cover of claim 9, wherein the sensor cover is reusable for a plurality of in vivo analyte sensors.

12. The analyte sensor cover of claim 9, wherein the adhesive component is substantially waterproof and sweatproof.

13. The analyte sensor cover of claim 9, wherein the adhesive component does not contact the in vivo analyte sensor.

14. The analyte sensor cover of claim 13, wherein the sensor cover permits the removal and replacement of the adhesive component without dislodging the in vivo analyte sensor.

15. The analyte sensor cover of claim 9, wherein the distance between the recessed portion and the annular rim is less than ⅛ inch.

16. The analyte sensor cover of claim 15, wherein the distance between the recessed portion and the annular rim is about 1/16 inch.

17. A system for retaining an in vivo analyte sensor in an embedded position, the system comprising:
a sensor cover including a top side and a bottom side and a first aperture, the bottom side comprised of a recessed portion and an annular rim around the recessed portion having a height to partially receive an in vivo analyte sensor;
a first aperture positioned at a central axis of the sensor cover to partially expose the in vivo analyte sensor to permit an interface between the in vivo analyte sensor and an analyte monitor;
an adhesive component adhered to the top side of the sensor cover and at least partially to the skin of a user and arranged to retain the in vivo analyte sensor in a suitable position, the adhesive component including a second aperture aligned with the first aperture;
a user performing the steps of:
embedding the in vivo analyte sensor;
positioning the sensor cover over the in vivo analyte sensor; and
adhering the adhesive component to the top surface of the sensor cover and the skin of the user to retain the in vivo analyte sensor in the suitable position.

18. The analyte sensor cover of claim 17, wherein the sensor cover permits the removal and replacement of the adhesive component without dislodging the in vivo analyte sensor.

19. The analyte sensor cover of claim 18, wherein the distance between the recessed portion and the annular rim is less than ⅛ inch.

20. The analyte sensor cover of claim 19, wherein the distance between the recessed portion and the annular rim is about 1/16 inch.
